SEQUENCE LISTING (2 sequences)

SEQ ID NO: 1 - RNA, 784 nt, synthetic construct
  source 1..784: mol_type = other RNA
aggaataaactagtattcttctggtccccacagactcagagagaacccgccaccatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaagaaggtgtataagactgtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaaaaaaaaaaaaaaaaaaaaaaaaaaaaaaaaaaaaaaaaa

SEQ ID NO: 2 - protein, 121 aa, synthetic construct
MNALQEDTPPGPSTVFRPPTSSRPLETPHCREIRIGIAGITITLSLCGCANARAPTLRSATADNSESTGFKNVPDLRTDQPKPPSKKRSCDPSEYRVSELKESLITTTPSRPRTARRCIRL

(19) United States
(12) Patent Application Publication
(10) Pub. No.: US 2026/0242423 A1
(43) Pub. Date: Aug. 20, 2026

(54) SYNTHETIC mRNA COMPRISING APOPTIN PROTEIN CODING SEQUENCE AND USE THEREOF

(71) Applicant: KNU-INDUSTRY COOPERATION FOUNDATION, Gangwon-do (KR)

(72) Inventors: HWAN WOO SEONG, Gangwon-do (KR); SUN YEOUNG SA, Gangwon-do (KR)

(21) Appl. No.: 19/162,197

(22) PCT Filed: Mar. 6, 2024
(86) PCT No.: PCT/KR2024/002830

§ 371 Date: Sep. 4, 2025

(30) Foreign Application Priority Data
May 18, 2023 (KR) 10-2023-0064071

(51) Int. Cl. C07K 14/005; A61K 38/00; A61P 35/00

(52) U.S. Cl. CPC C07K 14/005; A61P 35/00; A61K 38/00; C12N 2750/00022

(57) ABSTRACT

A synthetic mRNA includes an apoptin protein coding sequence and use thereof. The synthetic mRNA consisting of the nucleotide sequence of SEQ ID NO: 1 exhibits an excellent effect in inducing cancer cell death, and therefore, can be usefully applied in the field of development of anticancer therapeutic agents.

### REFERENCE TO AN ELECTRONIC SEQUENCE LISTING

A sequence listing electronically submitted on Sep. 4, 2025 as a XML file [1] named 20250904_S60225GR08_TU_SEQ.XML, created on Sep. 4, 2025 and having a size of 3,696 bytes, is incorporated herein by reference in its entirety.

### BACKGROUND

### 1. Technical Field

The present invention relates to a synthetic mRNA including apoptin protein coding sequence and use thereof.

### 2. Background Art

Apoptin is a protein derived from the chicken infectious anemia virus, composed of 121 amino acids, and is known to have cancer cell-specific apoptotic efficacy. While it does not induce apoptosis in normal cells, it exhibits selective cell death-inducing effects in various cancer cells, including liver cancer, lung cancer, hematologic cancer, and skin cancer. Because of this, various studies have been conducted to produce apoptin protein or develop gene vectors to deliver it for the development of anti-cancer therapeutics for humans and animals. However, due to the issues such as ineffective expression of high concentrations of apoptin protein in patients, there have been no reported cases to date of successful development of cancer therapeutics using apoptin.

Various studies using proteins like apoptin, which have cancer cell-killing efficacy, have been reported for the development of cancer therapeutics. Those studies mainly focus on methods for expressing such proteins with cancer cell-killing efficacy by using viral or bacterial expression vectors. However, when expressed in protein form, the yield is often insufficient for therapeutic use, and it is known that, when developed as a therapeutic, repeated administration (second or third doses) can lead to the formation of immune antibodies against the initially administered protein, resulting in problems like reduced therapeutic efficacy.

Meanwhile, Korean Patent Registration No. 0528755 discloses “Gene delivery vector expressing the cell apoptosis-inducing protein VP2 and/or apoptin using Adenoviral vector,” and Korean Patent Registration No. 0561985 discloses “Tumor-specific apoptin expression cassette and Adenovirus expression vector including the same.” However, there is no disclosure regarding the “Synthetic mRNA including apoptin protein coding sequence and use thereof” of the present invention.

### SUMMARY

The present invention is devised in response to the aforementioned needs. Inventors of the present invention have produced a synthetic mRNA in which 5′-cap, 5′-UTR (untranslated region), apoptin protein coding sequence, 3′-UTR, and poly(A) tail sequence are sequentially connected. Upon administering this synthetic mRNA to human-derived cancer cells (Hep-2 cells) and animal-derived cancer cells (DF-1 cells), respectively, the cell death rate resulting from treatment with the synthetic mRNA of the present invention was found to increase by approximately 2 to 3 times compared to the untreated control group. Through this finding, the present invention is completed.

To solve the problem described above, the present invention provides a synthetic mRNA for inhibiting the growth of or inducing the death of cancer cells, consisting of the nucleotide sequence of SEQ ID NO: 1.

The present invention further provides a method for producing a synthetic mRNA for inhibiting the growth of or inducing the death of cancer cells, including synthesizing an mRNA such that 5′-cap, 5′-UTR (untranslated region), apoptin protein coding sequence, 3′-UTR, and poly(A) tail sequence are sequentially connected.

The present invention further provides a pharmaceutical composition for prevention or treatment of cancer, including the synthetic mRNA as an active ingredient.

The present invention still further provides a functional health food composition for prevention or alleviation of cancer, including the synthetic mRNA as an active ingredient.

The synthetic mRNA according to the present invention exhibits an excellent effect in inducing cancer cell death and, therefore, can be usefully applied in the field of development of anticancer therapeutic agents.

### BRIEF DESCRIPTION OF THE DRAWINGS

FIGS. 1A and 1B show (FIG. 1A) a schematic diagram and (FIG. 1B) the nucleotide sequence of the synthetic mRNA of the present invention.

FIG. 2 shows the amino acid sequence translated from the apoptin protein coding sequence (KNU12014-F) included in the synthetic mRNA of the present invention.

FIGS. 3A and 3B present the results indicating the cancer cell killing effect of the synthetic mRNA of the present invention on human-derived cancer cells (Hep-2 cells).

In FIG. 3A, the cell death rate of the untreated control group is shown, while FIG. 3B shows the cell death rate of the test group treated with the synthetic mRNA of the present invention.

FIGS. 4A and 4B present the results indicating the cancer cell killing effect of the synthetic mRNA of the present invention on animal-derived cancer cells (DF-1 cells). In FIG. 4A, the cell death rate of the untreated control group is shown, while FIG. 4B shows the cell death rate of the test group treated with the synthetic mRNA of the present invention.

FIGS. 5A and 5B present the results indicating the cancer cell killing effect of the synthetic mRNA of the present invention in tumor-bearing BALB/c nude mice. In FIG. 5A, the changes in tumor volume in both the control group and the mRNA-treated group were measured over time up to 18 days after the injection of the synthetic mRNA, and then the results are compared between the two groups. FIG. 5B shows a comparison of tumor weights collected from the mice after sacrifice on 18 days after the injection.

### DETAILED DESCRIPTION

To achieve the objective of the present invention, the invention provides a synthetic mRNA for inhibiting the growth of or inducing the death of cancer cells, consisting of the nucleotide sequence of SEQ ID NO: 1.

The synthetic mRNA consisting of the nucleotide sequence of SEQ ID NO: 1 according to the present invention may be sequentially composed of 5′-cap, 5′-UTR (untranslated region), apoptin protein coding sequence, 3′-UTR, and poly(A) tail sequence. The apoptin protein preferably consists of the amino acid sequence of SEQ ID NO: 2, but is not limited thereto.

In the nucleotide sequence of the synthetic mRNA consisting of the nucleotide sequence of SEQ ID NO: 1 according to the present invention, uridine (U) may be substituted with N1-methylpseudouridine (m1ψ), although it is not limited thereto.

The present invention further provides a method for producing a synthetic mRNA for inhibiting the growth of or inducing the death of cancer cells, including synthesizing an mRNA such that 5′-cap, 5′-UTR (untranslated region), apoptin protein coding sequence, 3′-UTR, and poly(A) tail sequence are sequentially connected.

In the method for producing the synthetic mRNA of the present invention, the synthesis may be carried out according to techniques that are known to those skilled in the art and is not limited to any specific method.

The present invention further provides a pharmaceutical composition for prevention or treatment of cancer, including the synthetic mRNA of the present invention as an active ingredient.

In the pharmaceutical composition of the present invention, the synthetic mRNA is characterized by its anticancer effect through the induction of apoptosis in cancer cells.

The pharmaceutical composition of the present invention may further include suitable carriers, excipients, or diluents commonly used in the preparation of pharmaceutical compositions, and preferably may additionally include stabilizers such as liposomes, although it is not limited thereto.

The pharmaceutical dosage form of the composition according to the present invention may be used alone, in combination with other pharmaceutically active compounds, or as part of an appropriate formulation.

The pharmaceutical composition according to the present invention can be formulated into various dosage forms, such as oral formulations including powders, granules, tablets, capsules, suspensions, emulsions, syrups, and aerosols, as well as topical agents, suppositories, and sterile injectable solutions, according to conventional methods. The pharmaceutical composition containing the synthetic mRNA may include various carriers, excipients, and diluents such as lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methylparaben, propylparaben, talc, magnesium stearate, and mineral oil, among others, either alone or in combinations. When formulating, commonly used diluents or excipients such as fillers, extenders, binders, wetting agents, disintegrants, or surfactants are typically used. Solid oral formulations include tablets, pills, powders, granules, and capsules, which are prepared by mixing the synthetic mRNA with at least one excipient such as starch, calcium carbonate, sucrose or lactose, or gelatin. In addition to basic excipients, lubricants such as magnesium stearate and talc may also be used. Liquid oral formulations may include suspensions, internal solutions, emulsions, and syrups, which can contain not only simple diluents such as water and liquid paraffin, but also various excipients such as wetting agents, sweeteners, flavoring agents, and preservatives. Formulations for non-oral administration include sterile aqueous solutions, non-aqueous solvents, suspensions, emulsions, freeze-dried preparations, and suppositories. Non-aqueous solvents and suspensions may use injectable esters such as propylene glycol, polyethylene glycol, vegetable oils like olive oil, or ethyl oleate. Suppository bases may include Witepsol, macrogol, Tween 61, cacao butter, laurin butter, and glycerogelatin.

The preferred dosage of the pharmaceutical composition of the present invention varies depending on the patient's condition and weight, the severity of the disease, the dosage form, route of administration, and duration of treatment, but can be appropriately selected by those skilled in the art.

The present invention still further provides a functional health food composition for prevention or alleviation of cancer, including the synthetic mRNA of the present invention as an active ingredient.

The functional health food composition of the present invention may be formulated in any one form selected from powders, granules, pills, tablets, capsules, candies, syrups, and beverages, but is not limited thereto.

When the functional health food composition of the present invention is used as a food additive, it may be added directly or used together with other foods or food ingredients, and can be appropriately used according to conventional methods. The active ingredient may be used suitably according to its intended use (i.e., prevention or amelioration). Generally, when manufacturing foods or beverages, the functional health food composition of the present invention is added in an amount of 15 parts by weight or less, preferably 10 parts by weight or less, based on the raw materials. However, for long-term intake for health purposes, the amount may be below the stated range, and since there are no safety issues, the active ingredient may also be used in amounts exceeding the above range.

There is no particular limitation on the type of functional health food. Examples of foods to which the functional health food composition can be added include meat, sausages, bread, chocolate, candies, snacks, cookies, pizza, ramen, other noodles, gums, dairy products including ice cream, various soups, beverages, tea drinks, alcoholic beverages, and vitamin complexes, and encompass all health foods in the conventional sense.

The functional health food composition of the present invention can also be manufactured as a food, particularly as a functional food. The functional food of the present invention may include ingredients commonly added, such as proteins, carbohydrates, fats, nutrients, and seasonings. For example, when prepared as a drink, natural carbohydrates or flavoring agents may be included as additional ingredients besides the active ingredient. Preferred natural carbohydrates include monosaccharides (e.g., glucose, fructose), disaccharides (e.g., maltose, sucrose), oligosaccharides, polysaccharides (e.g., dextrin, cyclodextrin), or sugar alcohols (e.g., xylitol, sorbitol, erythritol). Flavoring agents may include natural flavorings (e.g., thaumatin, stevia extract) and synthetic flavorings (e.g., saccharin, aspartame).

In addition to the functional health food composition, various nutritional supplements, vitamins, electrolytes, flavor enhancers, colorants, pectic acid and its salts, alginic acid and its salts, organic acids, protective colloid thickeners, pH adjusters, stabilizers, preservatives, glycerin, alcohol, and carbonating agents used in carbonated beverages may also be included. Although the proportion of these additional components is not critically important, it is generally selected within the range of 0.01 to 0.1 parts by weight based on 100 parts by weight of the functional health food composition of the present invention.

Hereinafter, the present invention will be described in detail with reference to examples. However, the following examples are provided only to illustrate the present invention and are not intended to limit the scope of the invention to those examples.

### Example 1. Preparation of Synthetic mRNA

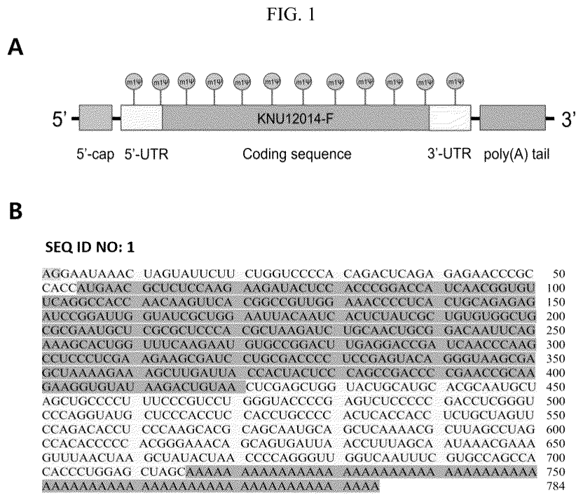

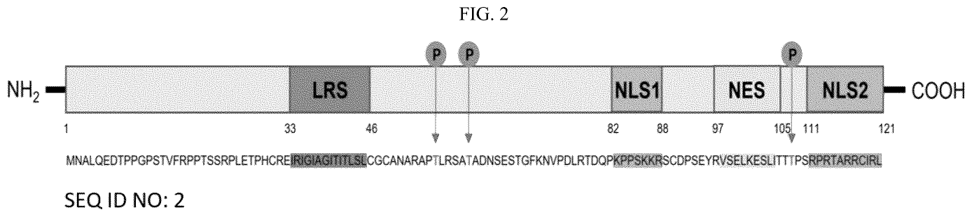

An IVT (in vitro transcribed) mRNA was synthesized in which, from the 5′ end, 5′-cap, 5′-UTR (untranslated region), apoptin protein (KNU12014-F) coding sequence, 3′-UTR, and poly(A) tail sequence are sequentially connected, and in the nucleotide sequence of the mRNA, all uridines (U) were replaced with N1-methylpseudouridine (m1ψ) during synthesis (FIGS. 1A and 1B). The 5′-UTR used was derived from human α-globin mRNA, and the 3′-UTR used was derived from mitochondrial 12S ribosomal RNA. The apoptin protein (KNU12014-F) coding sequence was cloned from the gene encoding VP3 protein of the KNU12014 strain of chicken infectious anemia virus, which the inventors of the present invention possess. Using synthetic DNA containing the 5′-cap, 5′-UTR, apoptin protein (KNU12014-F) coding sequence, 3′-UTR, and poly(A) tail sequence connected sequentially from the 5′ end as a template, mRNA was produced using an mRNA synthesis kit (HiScribe T7 mRNA Kit, NEB). The amino acid sequence translated from the apoptin protein coding sequence is composed of 121 amino acids, including LRS (leucine-rich sequence), NLS 1 (nuclear localization signal 1), NES (nuclear export signal), and NLS 2 (nuclear localization signal 2) (FIG. 2).

### Example 2. Analysis of Cancer Cell Killing Effect of Synthetic mRNA

To analyze the cancer cell killing effect of the synthetic mRNA according to the present invention, the cell death effects on human-derived cancer cells (Hep-2 cells, ATCC CCL-23) and animal-derived cancer cells (DF-1 cells, ATCC CRL-12203) were measured using flow cytometry.

Specifically, Hep-2 or DF-1 cells were seeded in 6-well plates at a concentration of 1×10⁶ cells per well and cultured for 24 hours to form a monolayer. Then, 100 ng of the synthetic mRNA from Example 1 were applied to each well, and the cells were cultured for 6 to 7 days. After incubation, the treated cells were trypsinized using 0.25% trypsin-EDTA, collected, and stained using an Annexin V-FITC Apoptosis Detection Kit (AB) according to the kit manual. Cell death was then determined by flow cytometry (Beckman Coulter).

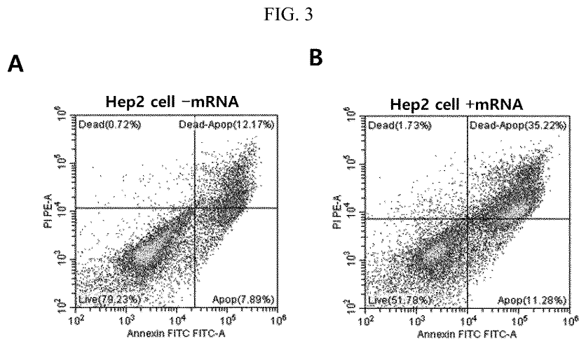

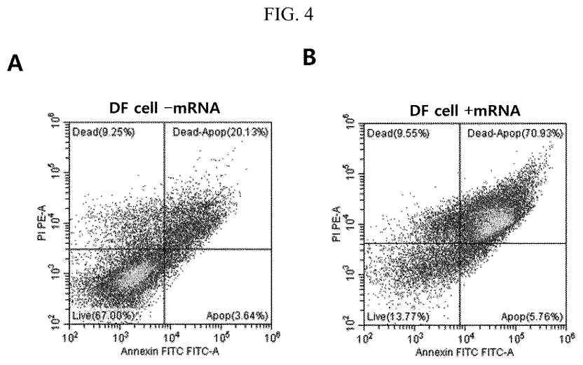

As a result, the cell death rate of human-derived Hep-2 cells treated with the synthetic mRNA of the present invention was found to be 46.5%, which is more than two times higher than that of the untreated control group (FIGS. 3A and 3B). It was also found that the cell death rate of animal-derived DF-1 cells treated with the synthetic mRNA was 76.69%, which is more than three times higher than that of the untreated control group (FIGS. 4A and 4B).

### Example 3. Analysis of Cancer Cell Killing Effect of Synthetic mRNA in Animal Model Expressing Cancer Cells

To evaluate the cancer cell killing effect of the synthetic mRNA according to the present invention in an animal model bearing cancer cells, human-derived Hep-2 cells (ATCC CCL-23) were xenografted into BALB/c nude mice, followed by administration of the synthetic mRNA to analyze the extent of cancer cell death.

Specifically, Hep-2 cells were inoculated into 4-week-old BALB/c nude mice (1×10⁶ Hep-2 cells per mouse) to establish tumors. Starting one week after inoculation, a mixture of Folate-Liposome (PEGylated, Creative Biolabs) and the synthetic mRNA of the present invention was administered intratumorally at a dose of approximately 5 μg per mouse every 3 to 4 days. Tumor volume and tumor weight were measured to compare the degree of cancer cell death. Tumor volume was calculated by measuring the long axis (a) and short axis (b) of the tumor and using the formula (a×b²×0.5). Tumor weight was measured by excising tumors from sacrificed BALB/c nude mice at the end of the study. The group treated with Folate-Liposome alone was used as the control.

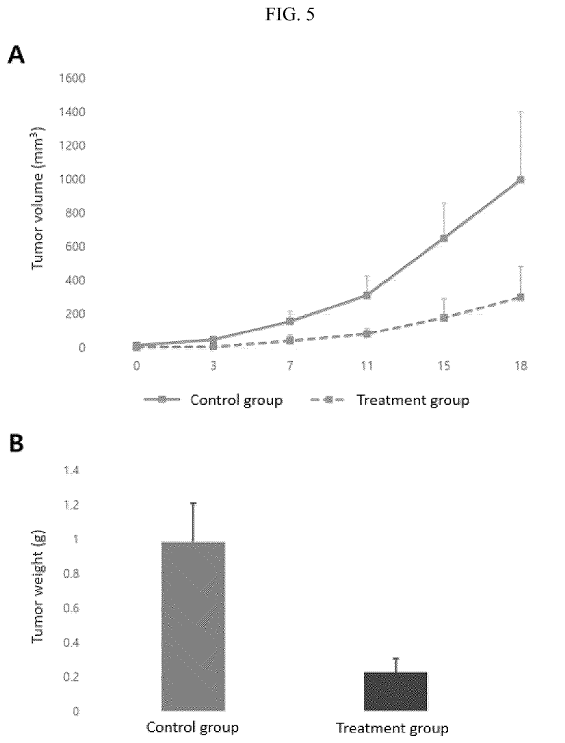

As a result, it was found that, from 7 days after the inoculation, tumor volume in the mRNA-treated group decreased compared to the control group. At 18 days after the inoculation, tumor volume in the mRNA-treated group (299±182 mm³) was reduced to 10 about 30% of that in the control group (996±404 mm³), and tumor weight in the treated group (0.22±0.08 g) was reduced to about 22% of the control group (0.99±0.22 g), demonstrating a significant reduction in tumor size (FIGS. 5A and 5B).

1. A synthetic mRNA consisting of the nucleotide sequence of SEQ ID NO: 1.

2. The synthetic mRNA according to claim 1, wherein the synthetic mRNA includes 5′-cap, 5′-UTR (untranslated region), apoptin protein coding sequence, 3′-UTR, and poly(A) tail sequence that are sequentially connected.

3. A method for producing a synthetic mRNA, comprising:
synthesizing an mRNA such that 5′-cap, 5′-UTR (untranslated region), apoptin protein coding sequence, 3′-UTR, and poly(A) tail sequence are sequentially connected.

4. A method for treatment of cancer, the method comprising:
administering a composition comprising the synthetic mRNA of claim 1 as an active ingredient to a subject in need thereof.

5. (canceled)

6. The method according to claim 4, wherein the composition further comprises a pharmaceutically acceptable carrier, excipient, and/or diluent.

7. The method according to claim 4, wherein the composition is prepared in any one formulation selected from capsule, powder, granule, tablet, suspension, emulsion, syrup, and aerosol.

8. The method of claim 4, wherein the composition is included in a functional health food.

9. The method of claim 8, wherein the functional health food is any one formulation selected from powder, granule, pill, tablet, capsule, candy, syrup, and beverage.
